(19) United States
(12) Patent Application Publication
(10) Pub. No.: US 2026/0363618 A1
(43) Pub. Date: Nov. 19, 2026

(54) ACCESSORY, MOBILE COMMUNICATION TERMINAL, PROGRAM, AND SERVER

(71) Applicant: E-LAMP. CO., LTD., Tokyo (JP)

(72) Inventors: Ayumi YAMAMOTO, Tokyo (JP); Shohei NISHIDA, Tokyo (JP)

(21) Appl. No.: 19/143,171

(22) PCT Filed: Dec. 26, 2022
(86) PCT No.: PCT/JP2022/048027

§ 371 Date: Jun. 25, 2025

(51) Int. Cl. A61B 5/00; A44C 7/00

(52) U.S. Cl. CPC A61B 5/6816; A44C 7/00

(57) ABSTRACT

An accessory and a mobile communication terminal that enable communication between users over a network through display of biological information are provided. An earring includes a sensor that detects the wearer's heart rate; LEDs serving as a display device; and a control unit. The control unit has a first operation mode and a second operation mode. In the first operation mode, the control unit controls the LEDs to display the wearer's heart rate. In the second operation mode, the control unit controls the LEDs based on another person's heart rate, or display manner information for another person, obtained from the outside.

### TECHNICAL FIELD

The present invention relates to accessories that display the wearers' biological information, and a system that includes the accessories.

### BACKGROUND ART

Accessories that display the wearers' biological information are known, including those disclosed in Patent Literature 1 and 2. The accessory disclosed in Patent Literature 1 is an earring to be worn on the wearer's ear. The earring includes a sensor that detects a pulse signal as biological information from the wearer's earlobe, an amplifier that amplifies the pulse signal, and a light-emitting body that emits light according to the amplified pulse signal.

The accessory disclosed in Patent Literature 2 is an earring to be worn on the wearer's ear. The earring includes a sensor that detects the wearer's heart rate as biological information from the wearer's earlobe; a display such as an LCD; a determination unit that determines the wearer's state such as a psychological or health state from the heart rate; and a display controller that controls the display content according to the wearer's state determined by the determination unit. The earring also includes a communication unit that connects to a mobile information terminal carried by the wearer. The mobile information terminal is used to configure operations of the earring. For example, the mobile information terminal is used to set an image to be displayed on the display.

### Citation List

### Patent Literature

Patent Literature 1
   Japanese Utility Model Publication No. H06-082913 A (1994-082913 A)
Patent Literature 2
   Japanese Patent No. 6815832 B

### SUMMARY OF INVENTION

### Technical Problem

As above, the accessory disclosed in Patent Literature 1 directly displays the wearer's biological information. The accessory disclosed in Patent Literature 2 indirectly displays the wearer's biological information in a manner that depends on the wearer's state. The wearer's biological information reflects the wearer's psychological state or emotions. Thus, the accessories disclosed in Patent Literature 1 and 2 are considered to have the function of conveying the wearer's psychological state or emotions.

By the way, in today's highly networked society, various tools allow mutual communication among people. A typical example of such tools is social media. However, the accessories disclosed in Patent Literature 1 and 2 are stand-alone devices. These accessories therefore cannot communicate the wearer's psychological state or emotions over a network. In addition, these accessories cannot appropriately display other people's psychological state or emotions.

The present invention has been made in view of the above circumstances, and an object thereof is to provide an accessory, a mobile communication terminal, a program, and a server that enable communication between users over a network through “display of biological information.”

### Solution to Problem

To achieve the above object, an accessory according to claim 1 of the present application includes: a sensor that detects biological information of a wearer of the accessory; an obtainment unit that obtains biological information of another person from an external source; a display unit; and a control unit having a first operation mode for controlling the display unit to display the biological information of the wearer, and a second operation mode for controlling the display unit to display at least the biological information of the other person.

An accessory according to claim 4 of the present application includes: a sensor that detects biological information of a wearer of the accessory; a storage unit that stores display manner information; a display unit; and a control unit having a first operation mode for controlling the display unit to display the biological information of the wearer according to display manner information for the wearer, and a second operation mode for controlling the display unit to display the biological information of the wearer according to second display manner information, wherein the second display manner information is display manner information for another person, or display manner information calculated from at least display manner information for another person.

A mobile communication terminal according to claim 11 of the present application includes a transfer unit that obtains biological information of another person from another mobile communication terminal via a network and transfers the biological information of the other person to the accessory according to claim 1 communicatively interconnected with the mobile communication terminal.

A mobile communication terminal according to claim 13 of the present application includes a setting unit that obtains display manner information for another person from another mobile communication terminal via a network and sets, for the accessory according to claim 4 communicatively interconnected with the mobile communication terminal, second display manner information derived from at least the display manner information for the other person.

A program according to claim 15 of the present application causes a computer to function as the transfer unit of the mobile communication terminal according to claim 11 or 12.

A program according to claim 16 of the present application causes a computer to function as the setting unit of the mobile communication terminal according to claim 13 or 14.

A server according to claim 17 of the present application includes a relay unit that relays biological information of another person between a plurality of the mobile communication terminals according to claim 11 or 12 via a network.

A server according to claim 18 of the present application includes a relay unit that relays display manner information for another person between a plurality of the mobile communication terminals according to claim 13 or 14 via a network.

### Advantageous Effects of Invention

In the accessory according to claim 1 of the present application, during the first operation mode of the control unit, the display unit displays the wearer's biological information. On the other hand, during the second operation mode of the control unit, the display unit displays another person's biological information obtained from an external source. That is, this enables communication between users through display of biological information.

In the accessory according to claim 4 of the present application, during the first operation mode of the control unit, the display unit displays the wearer's biological information according to display manner information for the wearer. On the other hand, during the second operation mode of the control unit, the display unit displays the wearer's biological information according to second display manner information. Here, the second display manner information is display manner information for another person, or display manner information calculated from at least display manner information for another person. Thus, this enables communication between users through display of biological information.

### BRIEF DESCRIPTION OF DRAWINGS

FIG. 1 is an overall configuration diagram of a communication system.

FIG. 2 is a side view of an earring, illustrating a vertical section of a case.

FIG. 3 is a block diagram of the earring.

FIG. 4 is a detailed block diagram of the earring and a mobile communication terminal.

FIG. 5 is a flowchart of a first operation mode.

FIG. 6 is a flowchart of coordination example 1 in a second operation mode.

FIG. 7 is a flowchart of coordination example 1 in the second operation mode.

FIG. 8 is a flowchart of coordination example 2 in the second operation mode.

FIG. 9 is a flowchart of coordination example 3 in the second operation mode.

FIG. 10 is a flowchart of coordination example 4 in the second operation mode.

FIG. 11 is a flowchart of coordination example 5 in the second operation mode.

FIG. 12 is a flowchart of coordination example 6 in the second operation mode.

### DESCRIPTION OF EMBODIMENTS

Accessories and a communication system including the accessories according to an embodiment of the present invention will be described with reference to the drawings.

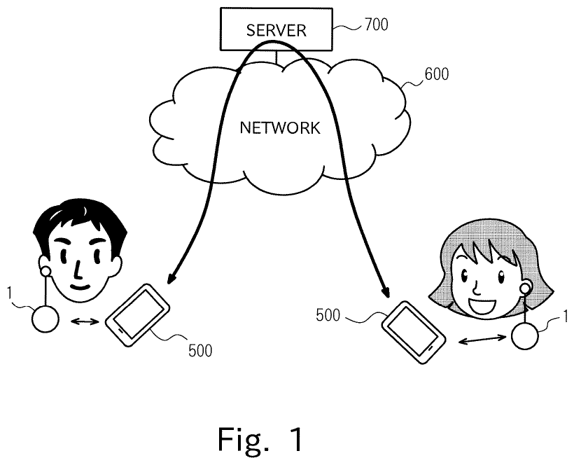

As illustrated in FIG. 1, a communication system including accessories according the embodiment includes: multiple pairs, each including an accessory 1 and a mobile communication terminal 500 carried by the wearer of the accessory 1; and a server 700 capable of communicating with the mobile communication terminals 500 over a network 600.

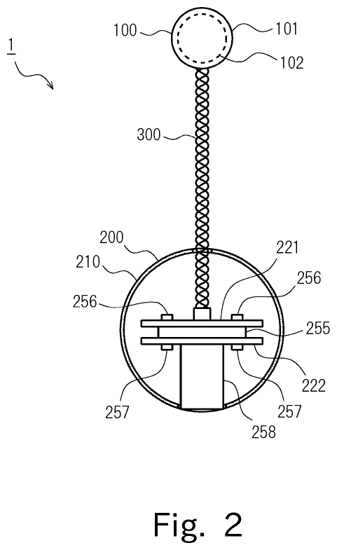

The following describes the accessories 1. In this example, the accessories are earrings. As illustrated in FIG. 2, each earring 1 includes an attachment 100 to be attached to an earlobe; a body 200; and a connector 300 that connects the attachment 100 and the body 200.

The attachment 100 includes a clip 101 to grip the earlobe, and a heartbeat sensor 102 disposed on the inner side of the clip 101. The heartbeat sensor 102 detects heartbeats as the wearer's biological information. The heartbeat sensor 102 is a known reflective heartbeat sensor having a light emitter and a light receiver. Light emitted from the light emitter toward the earlobe is reflected within the earlobe. The intensity of this reflected light changes with the blood flow (the intravascular volume) in the arteries within the earlobe. That is, the intensity of the reflected light depends on the pulsation of the heart. The heartbeat sensor 102 detects the intensity of the reflected light with the light receiver and outputs it as a heartbeat signal.

The body 200 includes a substantially spherical case 210 made of transparent or translucent resin, and a main board 221 and a sub-board 222 disposed in the case 210. The main board 221 and the sub-board 222 are stacked with a predetermined distance therebetween along the thickness direction. That is, the lower surface of the main board 221 and the upper surface of the sub-board 222 are facing each other. The main board 221 and the sub-board 222 are electrically connected.

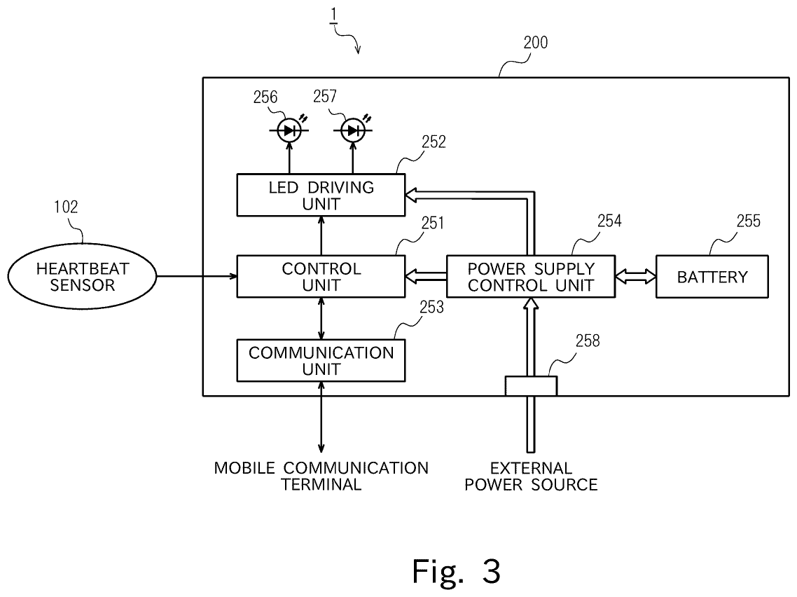

As illustrated in FIGS. 2 and 3, the main board 221 includes a control unit 251, an LED driving unit 252, a communication unit 253, a power supply control unit 254, a battery 255, and upper LEDs 256. The upper LEDs 256 are mounted on the upper surface of the main board 221. The battery 255 is mounted on the lower surface of the main board 221, i.e., between the main board 221 and the sub-board 222.

The sub-board 222 includes lower LEDs 257 and a charging connector 258. The lower LEDs 257 are mounted on the lower surface of the sub-board 222. The charging connector 258 is mounted on the lower surface of the sub-board 222. The charging connector 258 has a plug exposed on the bottom surface of the case 210.

The control unit 251 includes a microcomputer that integrates components, such as a CPU, a volatile main memory, a non-volatile secondary memory, an I/O interface, and a timer, into a single chip. The non-volatile secondary memory stores a control program. The non-volatile secondary memory also stores setting information to be described below. The control unit 251 functions by executing the control program. The details of the control unit 251 will be described below.

The LED driving unit 252 includes a circuit for driving the LEDs 256 and 257. The LEDs 256 and 257 function as a display device that displays biological information. The LED driving unit 252 amplifies control signals from the control unit 251 to drive the LEDs 256 and 257. The LEDs 256 and 257 can emit multiple colors of light. Specifically, the LEDs 256 and 257 include red, green, and blue light-emitting elements. Drive currents to the light-emitting elements of the different colors are controlled to enable the LEDs 256 and 257 to emit all colors of visible light. The colors of light emitted by the LEDs 256 and 257 are controlled by control signals from the control unit 251.

The communication unit 253 functions as an interface for wirelessly communicating with the mobile communication terminal 500 to be described below. In this example, the communication unit 253 complies with the Bluetooth (R) Low Energy standard.

The power supply control unit 254 supplies power from the battery 255 to the control unit 251, the LED driving unit 252, and the communication unit 253. The power supply control unit 254 also controls the charging of the battery 255 while an external power source is connected to the charging connector 258.

The connector 300 includes a cord-like or rod-like member that suspends the body 200 from the attachment 100. In this example, the connector 300 includes wires that electrically connect the heartbeat sensor 102 on the attachment 100 to the control unit 251 in the body 200. That is, the connector 300 has the function of physically connecting the body 200 and the attachment 100, and the function of electrically connecting them. In this example, the connector 300 includes a wire that supplies light-emission signals from the control unit 251 to the light emitter in the heartbeat sensor 102, a wire that transfers detection signals from the light receiver in the heartbeat sensor 102 to the control unit 251, and a ground wire. These three wires are twisted together. In addition to wires, the connector 300 may include a reinforcing cord-like or rod-like member.

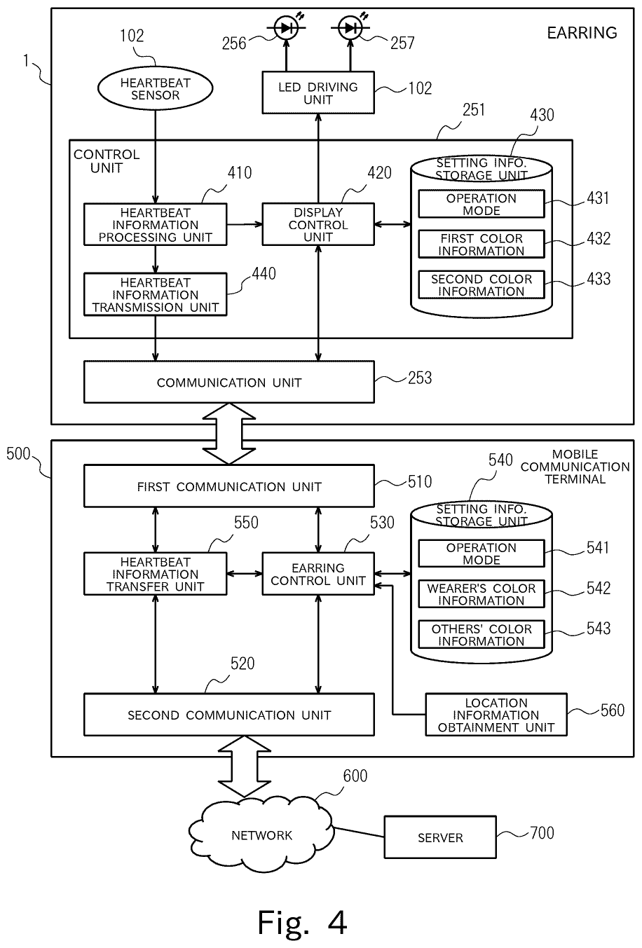

The earring 1 according to the present invention cooperates with the wearer's mobile communication terminal 500. The main role of the mobile communication terminal 500 is to configure settings of the earring 1, provide operation instructions to the earring 1, and transmit and receive various sorts of information to and from the outside. With reference to FIG. 4, the following describes a detailed configuration of the earring 1 and the mobile communication terminal 500.

The control unit 251 of the earring 1 includes a heartbeat information processing unit 410, a display control unit 420, a setting information storage unit 430, and a heartbeat information transmission unit 440.

The heartbeat information processing unit 410 calculates a heart rate per minute from a heartbeat signal detected by the heartbeat sensor 102. The heartbeat information processing unit 410 performs this calculation processing periodically, for example every second.

The display control unit 420 controls display on the LEDs 256 and 257 according to an operation mode 431 stored in the setting information storage unit 430. The details of the display control will be described below. The display control by the display control unit 420 starts and ends in response to a display start instruction and a display end instruction from the mobile communication terminal 500, respectively.

The setting information storage unit 430 stores the operation mode 431 as setting information. The operation mode 431 includes at least a first operation mode and a second operation mode. For the second operation mode, subordinate operation mode information may further be stored. The setting information storage unit 430 stores display manner information for determining the display manner for the display control on the LEDs 256 and 257. In this example, the display manner information includes light-emission color information for the LEDs 256 and 257. The setting information storage unit 430 stores first color information 432, which is display manner information for the wearer for use in the first operation mode, and second color information 433, which is second display manner information for use in the second operation mode. The information items stored in the setting information storage unit 430 are set with the mobile communication terminal 500.

The heartbeat information transmission unit 440 can transmit the heart rate calculated by the heartbeat information processing unit 410 to the mobile communication terminal 500.

The mobile communication terminal 500 includes a first communication unit 510 that communicates with the earring 1; a second communication unit 520 that connects to the network; an earring control unit 530 that controls the operations of the earring 1; a setting information storage unit 540; a heartbeat information transfer unit 550 that transfers heart rates; and a location information obtainment unit 560 that obtains location information of the mobile communication terminal 500.

The mobile communication terminal 500 is implemented by installing a program onto a mobile computer called a smartphone, which has communication functions, a display, a touch screen, and other components. The program installed on the computer causes the computer to function as at least the earring control unit 530, the setting information storage unit 540, the heartbeat information transfer unit 550, and the location information obtainment unit 560. The program is installed onto the computer via a predetermined storage medium or communication line.

The first communication unit 510 functions as an interface for wirelessly communicating with the communication unit 253 of the earring 1. In this example, the first communication unit 510 complies with the Bluetooth (R) Low Energy standard.

The second communication unit 520 functions as an interface for communicating with the server 700 over the network 600. The network 600, which is a communication path between the mobile communication terminal 500 and the server 700, may be in any configuration. Typically, the network 600 is configured by interconnecting one or more communication networks. If the network 600 includes a wireless communication network such as LTE, the second communication unit 520 includes a communication module for connecting to that wireless communication network. If the network 600 includes a wireless LAN, the second communication unit 520 includes an interface circuit for connecting to that wireless LAN.

The earring control unit 530 controls the settings of the earring 1, the operations of the earring 1, and the operations of the heartbeat information transfer unit 550, according to an operation mode 541 stored in the setting information storage unit 540. The detailed operations of the earring control unit 530 will be described below.

The setting information storage unit 540 stores the operation mode 541 of the earring 1 as setting information. The operation mode 541 is set by the wearer, who is the user of the mobile communication terminal 500. The operation mode 431 stored in the earring 1 is set by the earring control unit 530 copying the operation mode 541 in the setting information storage unit 540 to the operation mode 431.

The setting information storage unit 540 also stores display manner information for determining the display manner for the display control on the LEDs 256 and 257 of the earring 1. In this example, the display manner information includes light-emission color information for the LEDs 256 and 257. The setting information storage unit 540 stores the wearer's color information 542 for use in the first operation mode, and others' color information 543 for use in the second operation mode.

The heartbeat information transfer unit 550 transfers, to the earring 1 in response to an instruction from the earring control unit 530, another person's heart rate obtained from the outside via the network 600. The heartbeat information transfer unit 550 also transfers, to the outside via the network 600 in response to an instruction from the earring control unit 530, the heart rate obtained from the earring 1.

The location information obtainment unit 560 obtains location information of the mobile communication terminal 500. Methods of obtaining the location information include using a satellite positioning system, and using location information in a wireless communication network system.

The server 700 manages the users of the communication system and coordination among the users. The users of the communication system are the users of the pairs of earrings 1 and mobile communication terminals 500. The server 700 manages each user's account information, including at least identification information and password information. The server 700 also manages link information between users. For a given user, users linked with that user are referred to as the user's “friends.” The server 700 performs the processing of receiving a request from a user to be linked with another user, authenticating the request, and setting a link.

The server 700 also performs the processing of relaying information between mobile communication terminals 500 while the earring 1 is operating in the second operation mode to be described below. Note that the relay processing may be performed only at the start of display operation of the earring 1, or may be performed in real time during the operation of the earring 1. In the latter case, the server 700 may establish a communication path between the mobile communication terminals 500 and instruct them to perform peer-to-peer data communication.

The earring control unit 530 of the mobile communication terminal 500 receives an input of the wearer's color information 542 in advance from the user and stores the wearer's color information 542 in the setting information storage unit 540. The earring control unit 530 receives an input of the operation mode 541 from the user and stores the operation mode 541 in the setting information storage unit 540. Here, if the second operation mode is selected as the operation mode 541, the earring control unit 530 can obtain, via the server 700, color information of another person who is a friend of the user, and store the other person's color information as the others' color information 543 in the setting information storage unit 540.

Upon receiving an input of an instruction to start display operation from the user, the earring control unit 530 sets the operation mode 431 in the setting information storage unit 430 of the earring 1 to the operation mode 541 stored in the setting information storage unit 540. The earring control unit 530 can also set the first color information 432 in the setting information storage unit 430 of the earring 1 to the wearer's color information 542 stored in the setting information storage unit 540. If the second operation mode is selected as the operation mode 541, the earring control unit 530 can also derive second color information and set this second color information as the second color information 433 in the setting information storage unit 430 of the earring 1. The earring control unit 530 instructs, according to the instruction from the user, the display control unit 420 of the earring 1 to start display operation.

If the second operation mode is selected as the operation mode 541, the earring control unit 530 can periodically re-derive the second color information during the operation of the earring 1 to update the second color information 433 in the setting information storage unit 430 of the earring 1.

If the second operation mode is selected as the operation mode 541, the earring control unit 530 can obtain the friend's heartbeat information in real time via the server 700 during the operation of the earring 1. If the second operation mode is selected as the operation mode 541, the earring control unit 530 can instruct the heartbeat information transfer unit 550 to transfer the friend's heartbeat information obtained via the server 700 to the earring 1 in real time during the operation of the earring 1.

If the second operation mode is selected as the operation mode 541, the earring control unit 530 can obtain the wearer's heartbeat information in real time from the earring 1 during the operation of the earring 1. If the second operation mode is selected as the operation mode 541, the earring control unit 530 can instruct the heartbeat information transfer unit 550 to transfer the wearer's heartbeat information obtained from the earring 1 to the friend's mobile communication terminal 500 via the server 700 during the operation of the earring 1.

The display control unit 420 of the earring 1, upon receiving the display operation start instruction from the earring control unit 530, performs the display control on the LEDs 256 and 257 according to the operation mode 431 stored in the setting information storage unit 430.

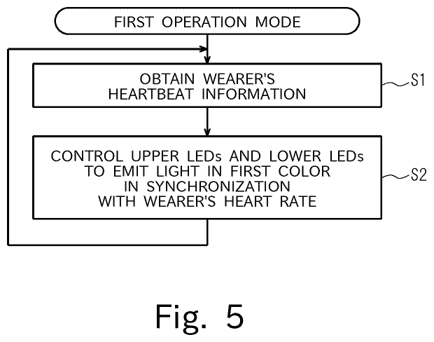

Operations in the first operation mode will be described. In the first operation mode, the earring 1 operates based on the wearer's heart rate and the first color information 432, which is the display manner information for the wearer. That is, in the first operation mode, the earring 1 operates on a stand-alone basis. Specifically, as illustrated in FIG. 5, the display control unit 420 obtains the wearer's heart rate from the heartbeat information processing unit 410 (step S1) and controls the LEDs 256 and 257 to flash in synchronization with the wearer's heart rate (step S2). Here, the display control unit 420 controls the LEDs 256 and 257 to emit light in the color specified by the first color information 432. Note that the color specified by the first color information 432 corresponds to the wearer's color information 542.

Now, operations in the second operation mode will be described. In the second operation mode, the earring 1 operates in coordination with information items on a friend. Information necessary for the coordination is relayed by data communication between mobile communication terminals 500 via the server 700. Various examples of coordination manners can be illustrated. The following describes coordination examples in the second operation mode.

### 1. Coordination With the Friend's Heart Rate

### Operations on the Transmitter Side

First, the earring 1 and the mobile communication terminal 500 on the data transmitter side will be described. The earring control unit 530 of the mobile communication terminal 500 on the transmitter side instructs the heartbeat information transmission unit 440 of the earring 1 to transmit the wearer's heart rate to the mobile communication terminal 500. The earring control unit 530 further instructs the heartbeat information transfer unit 550 to transfer the wearer's heartbeat information obtained from the earring 1 to the friend's mobile communication terminal 500 via the server 700.

### Operations on the Receiver Side

In this coordination operation, the second operation mode further includes two subordinate operation modes. A first subordinate operation mode is “full light mode,” and a second subordinate operation mode is “half-and-half light mode.” The setting of subordinate operation mode is made when the operation mode is set on the mobile communication terminal 500 as described above.

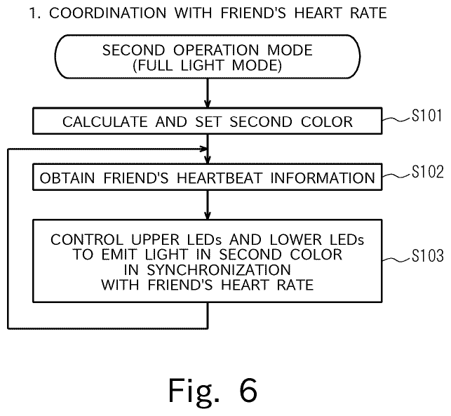

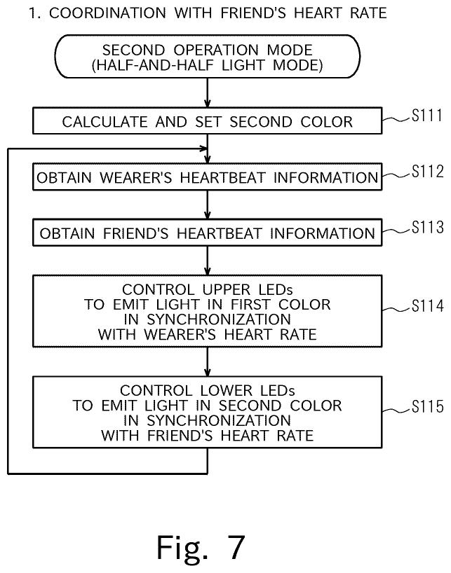

As illustrated in FIGS. 6 and 7, the earring control unit 530 of the mobile communication terminal 500 on the receiver side derives, as the second color information, a color distinguishable from the color corresponding to the wearer's color information 542, and sets this second color information as the second color information 433 in the setting information storage unit 430 of the earring 1 (steps S101 and S111). The second color information is, for example, the complementary color of the color corresponding to the wearer's color information 542. The earring control unit 530 then instructs the heartbeat information transfer unit 550 to transfer the friend's heartbeat information obtained via the server 700 to the earring 1 in real time.

In full light mode, as illustrated in FIG. 6, the display control unit 420 of the earring 1 obtains the friend's heart rate from the mobile communication terminal 500 (step S102) and controls the upper LEDs 256 and the lower LEDs 257 to flash in synchronization with the friend's heart rate (step S103). Here, the earring control unit 530 controls the upper LEDs 256 and the lower LEDs 256 to emit light in the color specified by the second color information 433.

In half-and-half light mode, as illustrated in FIG. 7, the display control unit 420 of the earring 1 obtains the wearer's heart rate from the heartbeat information processing unit 410, and obtains the friend's heart rate from the mobile communication terminal 500 (steps S112 and S113). The display control unit 420 controls the upper LEDs 256 to flash in synchronization with the wearer's heart rate, and controls the lower LEDs 257 to flash in synchronization with the friend's heart rate. Here, the earring control unit 530 controls the upper LEDs 256 to emit light in the color specified by the first color information 432, and controls the lower LEDs 257 to emit light in the color specified by the second color information 433 (steps S114 and S115). Note that the color specified by the first color information 432 corresponds to the wearer's color information 542.

Through the above process, in full light mode, the friend's heart rate is displayed in a color different from the wearer's color on the LEDs 256 and 257 of the wearer's earring 1. In half-and-half light mode, the friend's heart rate is displayed in a color different from the wearer's color on the lower LEDs 257 of the wearer's earring 1.

### 2. Coordination With the Friend's Color Information

### Operations on the Transmitter Side

First, the earring 1 and the mobile communication terminal 500 on the data transmitter side will be described. The earring control unit 530 of the mobile communication terminal 500 on the transmitter side transfers the wearer's color information 542 to the friend's mobile communication terminal 500 via the server 700.

### Operations on the Receiver Side

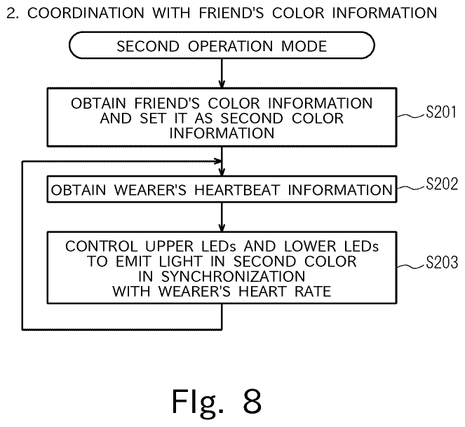

As illustrated in FIG. 8, the earring control unit 530 of the mobile communication terminal 500 on the receiver side obtains the friend's color information from the friend's mobile communication terminal 500 via the server 700, and sets this color information as the second color information 433 in the setting information storage unit 430 of the earring 1 (step S201).

The display control unit 420 of the earring 1 obtains the wearer's heart rate from the heartbeat information processing unit 410, and controls the upper LEDs 256 and the lower LEDs 257 to flash in synchronization with the wearer's heart rate (steps S202 and S203). Here, the earring control unit 530 controls the upper LEDs 256 and the lower LEDs 257 to emit light in the color specified by the second information 433, i.e., the friend's color information.

Through the above process, the wearer's heart rate is displayed in the friend's color on the LEDs 256 and 257 of the wearer's earring 1. Note that, in this coordination example, half-and-half light mode may be provided as in [1. Coordination with the friend's heart rate].

### 3. Coordination With the Friend's Heart Rate and Color Information (1)

### Operations on the Transmitter Side

First, the earring 1 and the mobile communication terminal 500 on the data transmitter side will be described. The earring control unit 530 of the mobile communication terminal 500 on the transmitter side instructs the heartbeat information transmission unit 440 of the earring 1 to transmit the wearer's heart rate to the mobile communication terminal 500. The earring control unit 530 further instructs the heartbeat information transfer unit 550 to transfer the wearer's heartbeat information obtained from the earring 1 to the friend's mobile communication terminal 500 via the server 700. The earring control unit 530 also transfers the wearer's color information 542 to the friend's mobile communication terminal 500 via the server 700.

### Operations on the Receiver Side

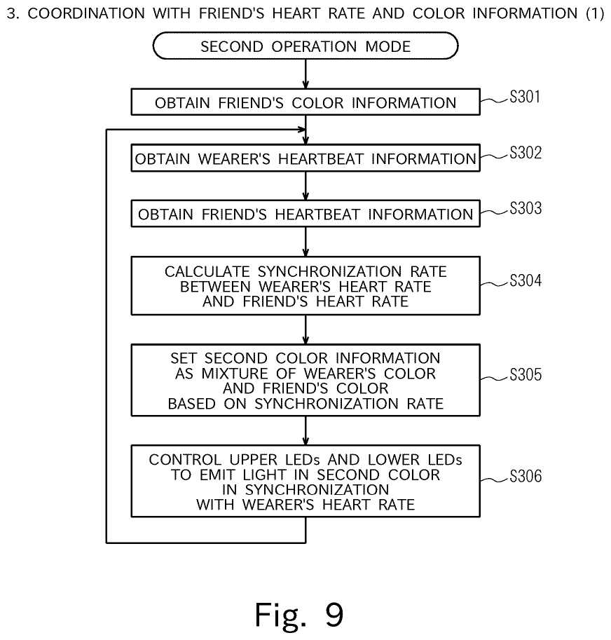

As illustrated in FIG. 9, the earring control unit 530 of the mobile communication terminal 500 on the receiver side obtains the friend's color information from the friend's mobile communication terminal 500 via the server 700, and stores this color information as the others' color information 543 in the setting information storage unit 540 (step S301). The earring control unit 530 also instructs the heartbeat information transmission unit 440 of the earring 1 to transmit the wearer's heart rate to the mobile communication terminal 500.

The earring control unit 530 then obtains the wearer's heart rate from the earring 1 and obtains the friend's heart rate from the server 700 (steps S302 and S303). The earring control unit 530 then calculates the second color information from the wearer's heart rate, the friend's heart rate, the wearer's color information 542, and the others' color information 543, i.e., the friend's color information. The earring control unit 530 sets this second color information as the second color information 433 in the setting information storage unit 430 of the earring 1 (steps S304 and S305).

The following describes the calculation of the second color information. The earring control unit 530 calculates the synchronization rate between the wearer's heart rate and the friend's heart rate (step S304). Here, an example of calculating the synchronization rate involves calculating the synchronization rate from the difference between the wearer's heart rate and the friend's heart rate. The synchronization rate is calculated using a function such that the synchronization rate increases as the difference decreases, and the synchronization rate decreases as the difference increases. Here, the lower and upper limits for the difference may be defined. The function may be linear or nonlinear. For example, the synchronization rate may be 100% for differences smaller than or equal to a predetermined lower limit value (e.g., 5 bpm), and 0% for differences greater than or equal to a predetermined upper limit value (e.g., 50 bpm).

Another example of calculating the synchronization rate involves calculating the difference between the rate of change per unit time in the wearer's heart rate and the rate of change per unit time in the friend's heart rate. The synchronization rate is calculated using a function such that the synchronization rate increases as the difference decreases, and the synchronization rate decreases as the difference increases. Here, the lower and upper limits for the difference may be defined. The function may be linear or nonlinear. For example, the synchronization rate may be 100% for differences smaller than or equal to a predetermined lower limit value (e. g., 1 bpm/sec), and 0% for differences greater than or equal to a predetermined upper limit value (e.g., 5 bpm/sec).

The earring control unit 530 then calculates the second color information by mixing “the color corresponding to the wearer's color information 542” and “the color corresponding to the others' color information 543, i.e., the friend's color information, multiplied by the above synchronization rate” (step S305). Thus, for a synchronization rate of 0%, the color corresponding to the second color information is the color corresponding to the wearer's color information 542. For a synchronization rate of 100%, the color corresponding to the second color information is the color right between the color corresponding to the wearer's color information 542 and the color corresponding to the others' color information 543, i.e., the friend's color information.

The earring control unit 530 continuously obtains the wearer's heart rate and the friend's heart rate to calculate and update the second color information 433.

The display control unit 420 of the earring 1 obtains the wearer's heart rate from the heartbeat information processing unit 410, and controls the upper LEDs 256 and the lower LEDs 257 to flash in synchronization with the wearer's heart rate (step S306). Here, the earring control unit 530 controls the upper LEDs 256 and the lower LEDs 257 to emit light in the color specified by the second color information 433.

Through the above process, the wearer's heart rate is displayed on the LEDs 256 and 257 of the wearer's earring 1 in a color that is a mixture of the wearer's color and the friend's color. The degree of mixture of the display color varies with the synchronization rate between the wearer's heart rate and the friend's heart rate.

### 4. Coordination With the Friend's Heart Rate and Color Information (2)

### Operations on the Transmitter Side

First, the earring 1 and the mobile communication terminal 500 on the data transmitter side will be described. The earring control unit 530 of the mobile communication terminal 500 on the transmitter side instructs the heartbeat information transmission unit 440 of the earring 1 to transmit the wearer's heart rate to the mobile communication terminal 500. The earring control unit 530 further instructs the heartbeat information transfer unit 550 to transfer the wearer's heartbeat information obtained from the earring 1 to the friend's mobile communication terminal 500 via the server 700. The earring control unit 530 also transfers the wearer's color information 542 to the friend's mobile communication terminal 500 via the server 700.

### Operations on the Receiver Side

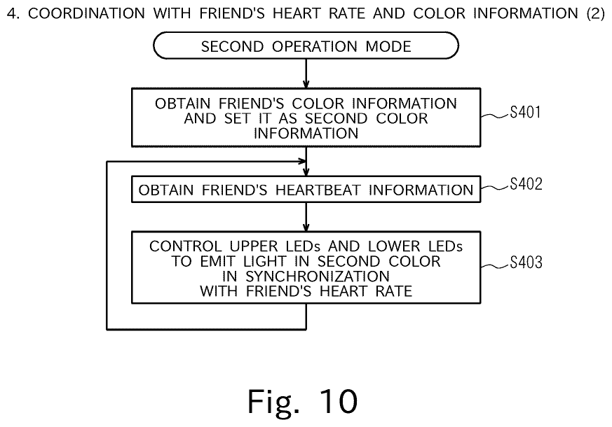

As illustrated in FIG. 10, the earring control unit 530 of the mobile communication terminal 500 on the receiver side obtains the friend's color information from the friend's mobile communication terminal 500 via the server 700, stores this color information as the others' color information 543 in the setting information storage unit 540, and also sets this color information as the second color information 433 in the setting information storage unit 430 of the earring 1 (step S401). The earring control unit 530 instructs the heartbeat information transfer unit 550 to transfer the friend's heartbeat information obtained via the server 700 to the earring 1 in real time.

The display control unit 420 of the earring 1 obtains the friend's heart rate from the mobile communication terminal 500, and controls the upper LEDs 256 and the lower LEDs 257 to flash in synchronization with the friend's heart rate (steps S402 and S403). Here, the earring control unit 530 controls the upper LEDs 256 and the lower LEDs 257 to emit light in the color specified by the second color information 433.

Through the above process, the friend's heart rate is displayed in the friend's color on the LEDs 256 and 257 of the wearer's earring 1. Note that, in this coordination example, half-and-half light mode may be provided as in [1. Coordination with the friend's heart rate].

### 5. Coordination With the Friend's Heart Rate and Color Information (3)

This coordination example involves coordinating with the heart rate and color information in relation to multiple friends.

### Operations on the Transmitter Side

First, the earring 1 and the mobile communication terminal 500 on each of multiple data transmitter sides will be described. The earring control unit 530 of the mobile communication terminal 500 on the transmitter side instructs the heartbeat information transmission unit 440 of the earring 1 to transmit the wearer's heart rate to the mobile communication terminal 500. The earring control unit 530 further instructs the heartbeat information transfer unit 550 to transfer the wearer's heartbeat information obtained from the earring 1 to the friend's mobile communication terminal 500 via the server 700. The earring control unit 530 also transfers the wearer's color information 542 to the friend's mobile communication terminal 500 via the server 700.

### Operations on the Receiver Side

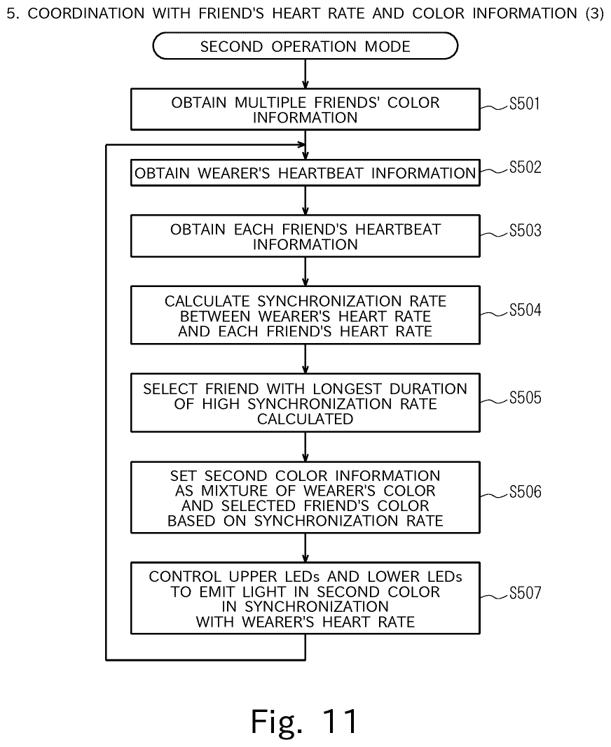

As illustrated in FIG. 11, the earring control unit 530 of the mobile communication terminal 500 on the receiver side obtains each friend's color information from the friend's mobile communication terminal 500 via the server 700, and stores this color information as the others' color information 543 in the setting information storage unit 540 (step S501). The earring control unit 530 also instructs the heartbeat information transmission unit 440 of the earring 1 to transmit the wearer's heart rate to the mobile communication terminal 500.

The earring control unit 530 then obtains the wearer's heart rate from the earring 1 and obtains each friend's heart rate from the server 700 (steps S502 and S503). The earring control unit 530 then calculates the second color information from the wearer's heart rate, the friend's heart rate, the wearer's color information 542, and the others' color information 543, i.e., the friend's color information. The earring control unit 530 sets this second color information as the second color information 433 in the setting information storage unit 430 of the earring 1 (steps S503 to S506).

The following describes the calculation of the second color information. For each friend, the earring control unit 530 calculates the synchronization rate between the wearer's heart rate and the friend's heart rate (step S504). Here, an example of calculating the synchronization rate involves calculating the synchronization rate from the difference between the wearer's heart rate and the friend's heart rate. The synchronization rate is calculated using a function such that the synchronization rate increases as the difference decreases, and the synchronization rate decreases as the difference increases. Here, the lower and upper limits for the difference may be defined. The function may be linear or nonlinear. For example, the synchronization rate may be 100% for differences smaller than or equal to a predetermined lower limit value (e.g., 5 bpm), and 0% for differences greater than or equal to a predetermined upper limit value (e.g., 50 bpm).

Another example of calculating the synchronization rate involves calculating the difference between the rate of change per unit time in the wearer's heart rate and the rate of change per unit time in the friend's heart rate. The synchronization rate is calculated using a function such that the synchronization rate increases as the difference decreases, and the synchronization rate decreases as the difference increases. Here, the lower and upper limits for the difference may be defined. The function may be linear or nonlinear. For example, the synchronization rate may be 100% for differences smaller than or equal to a predetermined lower limit value (e.g., 1 bpm/sec), and 0% for differences greater than or equal to a predetermined upper limit value (e.g., 5 bpm/sec).

The earring control unit 530 then selects, among the friends, the friend with the longest cumulative duration of a high synchronization rate calculated within a predetermined period (step S505). Here, a high synchronization rate refers to a synchronization rate higher than a threshold. The threshold may be a predetermined static value. The threshold may also be a value calculated statistically from the friends' synchronization rates at a certain timepoint. Alternatively, a high synchronization rate may be the synchronization rate of the friend with the Nth highest synchronization rate, where N is a natural number. For N=1, the friend with the longest cumulative duration of the synchronization rate ranked first is selected.

The earring control unit 530 then calculates the second color information by mixing “the color corresponding to the wearer's color information 542” and “the color corresponding to the selected friend's color information 543, multiplied by the selected friend's synchronization rate” (step S506). Thus, for a synchronization rate of 0%, the color corresponding to the second color information is the color corresponding to the wearer's color information 542. For a synchronization rate of 100%, the color corresponding to the second color information is the color right between the color corresponding to the wearer's color information 542 and the color corresponding to the selected friend's color information in the others' color information 543.

The earring control unit 530 continuously obtains the wearer's heart rate and each friend's heart rate to calculate and update the second color information 433.

The display control unit 420 of the earring 1 obtains the wearer's heart rate from the heartbeat information processing unit 410, and controls the upper LEDs 256 and the lower LEDs 257 to flash in synchronization with the wearer's heart rate (step S507). Here, the earring control unit 530 controls the upper LEDs 256 and the lower LEDs 257 to emit light in the color specified by the second color information 433.

Through the above process, the wearer's heart rate is displayed on the LEDs 256 and 257 of the wearer's earring 1 in a color that is a mixture of the wearer's color and the friend's color. Here, the friend whose color is mixed is the friend with the longest cumulative duration of a high synchronization rate calculated within a predetermined period, among the multiple friends. In addition, the degree of mixture of the display color varies with the synchronization rate between the wearer's heart rate and the friend's heart rate.

### 6. Coordination With the Friend's Location Information and Color Information

### Operations on the Transmitter Side

First, the earring 1 and the mobile communication terminal 500 on the data transmitter side will be described. The earring control unit 530 of the mobile communication terminal 500 on the transmitter side obtains the wearer's location information from the location information obtainment unit 560, and transmits the location information to the friend's mobile communication terminal 500 via the server 700 in real time. The earring control unit 530 also transfers the wearer's color information 542 to the friend's mobile communication terminal 500 via the server 700.

### Operations on the Receiver Side

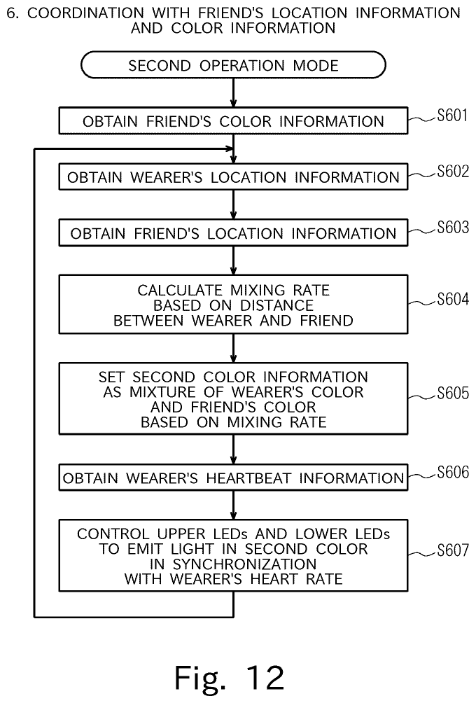

As illustrated in FIG. 12, the earring control unit 530 of the mobile communication terminal 500 on the receiver side obtains the friend's color information from the friend's mobile communication terminal 500 via the server 700, and stores this color information as the others' color information 543 in the setting information storage unit 540 (step S601).

The earring control unit 530 then obtains the wearer's location information from the location information obtainment unit 560 and obtains the friend's location information from the server 700 (steps S602 and S603). The earring control unit 530 then calculates the second color information from the wearer's location information, the friend's location information, the wearer's color information 542, and the others' color information 543, i.e., the friend's color information. The earring control unit 530 sets this second color information as the second color information 433 in the setting information storage unit 430 of the earring 1 (steps S604 and S605).

The following describes the calculation of the second color information. The earring control unit 530 calculates the distance between the wearer and the friend based on the wearer's location information and the friend's location information. The earring control unit 530 then calculates a mixing rate based on the distance (step S604). Here, the mixing rate is calculated using a function such that the mixing rate increases as the distance decreases, and the mixing rate decreases as the distance increases. The lower and upper limits for the distance may also be defined. The function may be linear or nonlinear. For example, the mixing rate may be 100% for distances shorter than or equal to a predetermined lower limit value (e. g., 10 m), and 0% for distances longer than or equal to a predetermined upper limit value (e.g., 10 km).

The earring control unit 530 then calculates the second color information by mixing “the color corresponding to the wearer's color information 542” and “the color corresponding to the others' color information 543, i.e., the friend's color information, multiplied by the mixing rate” (step S605). Thus, for a mixing rate of 0%, the color corresponding to the second color information is the color corresponding to the wearer's color information 542. For a mixing rate of 100%, the color corresponding to the second color information is the color right between the color corresponding to the wearer's color information 542 and the color corresponding to the others' color information 543, i.e., the friends color information.

The earring control unit 530 continuously obtains the wearer's location information and the friend's location information to calculate and set the second color information 433.

The display control unit 420 of the earring 1 obtains the wearer's heart rate from the heartbeat information processing unit 410, and controls the upper LEDs 256 and the lower LEDs 257 to flash in synchronization with the wearer's heart rate (steps S606 and S607). Here, the earring control unit 530 controls the upper LEDs 256 and the lower LEDs 257 to emit light in the color specified by the second color information 433.

Through the above process, the wearer's heart rate is displayed on the LEDs 256 and 257 of the wearer's earring 1 in a color that is a mixture of the wearer's color and the friend's color. The degree of mixture of the display color then varies with the distance between the wearer and the friend.

Thus, the earring 1 according to this example enables a user to use the mobile communication terminal 500 to obtain information items on the user's friend, such as heartbeat information, color information, and location information, which can then be used to display the heartbeat information in various display forms. That is, multiple users, each having a pair of earring 1 and mobile communication terminal 500, can communicate with each other over a network through “display of biological information.”

Now, other applications of the communication system including the earrings 1 according to this example will be described.

As described above, the pairs of earrings 1 and mobile communication terminals 500 according to this example are capable of bidirectional communication of information items. In other applications, multiple earrings 1 and mobile communication terminals 500 may receive common information items unidirectionally from a predetermined distributor in the second operation mode. The distributor may be a specific earring 1 and mobile communication terminal 500 according to this example. Alternatively, the distributor may be some other device having the function of obtaining and distributing information items such as heartbeat information, color information, and location information.

A specific example of such applications is the use of the system at a concert venue. In this case, the distributor is each of performers. Users, who are the audience, can each set one of the performers as a friend. The server 700 managing friends preferably presents the concert performers as friend candidates to the users to facilitate the users' friend setting.

Another application is the use of the system in a matchmaking party. In this case, the second operation mode for the earrings 1 and the mobile communication terminals 500 corresponds to a modification of [5. Coordination with the friend's heart rate and color information (3)]. That is, in the above coordination example, each user's color information and friends are set by the user. In contrast, in this application, the server 700 managing friends selects, for each user, participants of the opposite sex from among the party participants as the user's friends and sets the color information of all the participants so that it does not overlap within the same sex. Each participant's mobile communication terminal 500 can access the server 700 to set the selected participants as the participant's friends and set the specified color information as the participant's color information.

Thus, an embodiment of the present invention has been described. The present invention is not limited to the above embodiment, and various improvements and modifications may be made without departing from the spirit of the present invention.

For example, the earrings described as exemplary accessories in the above embodiment may be replaced by other types of accessories. Other exemplary accessories include finger rings, bracelets, neck rings, anklets, necklaces, hair ornaments, hats, glasses, sunglasses, earphones, headphones, watches, pocket watches, straps, key chains, and badges. In the above embodiment, each earring includes the connector that connects the clip having the sensor to the body having the display unit. Alternatively, the clip and the body may be integrally formed.

In the above embodiment, the sensor is incorporated in the earring. Alternatively, depending on the accessory type and the sensor type, the sensor may be an article separate from the accessory body having the display unit. In this case, the sensor and the accessory body can be attached to different positions on the wearer's body. For example, if the accessory is a strap, the strap body having the display unit may be attached to the wearer's bag, whereas the sensor that detects heartbeats can be attached to the wearer's ear or arm. The T sensor and the accessory body having the display unit implemented as separate articles as above may also be connected via wireless or wired connection.

The heartbeat information described as the biological information in the above embodiment may also be replaced by other types of biological information. The biological information is preferably information that changes with the wearer's physical or mental state. Exemplary biological information includes facial expressions, body movements, body temperature, electromyogram, electrocardiogram, brain waves, blood pressure, respiratory rate, perspiration, and pupil movements. Multiple types of biological information may also be combined.

In the above embodiment, the heart rate is displayed as the biological information by flashing the LEDs in synchronization with the heart rate. Alternatively, the heart rate may be displayed in other ways. For example, a seven-segment LED, LCD, or organic EL panel may be used as the display device to display the heart rate as a numerical value. Alternatively, an image corresponding to the heart rate may be displayed. In the above embodiment, the LED display color information is used as the display manner information for displaying the heart rate as the biological information. However, other types of display manner information may be used, depending on the method or device for displaying the biological information. For example, if the display device is an organic EL panel, the display manner information may be display color information or an image.

In the above embodiment, the second color information for use in the second operation mode is calculated by the mobile communication terminal. Alternatively, the second color information may be calculated by the accessory. The accessory may also have functions of the mobile communication terminal.

### REFERENCE SIGNS LIST

1 earring
100 attachment
101 clip
102 heartbeat sensor
200 body
210 case
251 control unit
252 LED driving unit
253 communication unit
254 power supply control unit
255 battery
256 upper LED
257 lower LED
258 charging connector
410 heartbeat information processing unit
420 display control unit
430 setting information storage unit
440 heartbeat information transmission unit
500 mobile communication terminal
510 first communication unit
520 second communication unit
530 earring control unit
540 setting information storage unit
550 heartbeat information transfer unit
600 network
700 server

1. An accessory comprising:
a sensor that detects biological information of a wearer of the accessory;
an obtainment unit that obtains biological information of another person from an external source;
a display unit; and
a control unit having a first operation mode for controlling the display unit to display the biological information of the wearer, and a second operation mode for controlling the display unit to display at least the biological information of the other person, wherein
the display unit comprises a first display element and a second display element, and
in the second operation mode, the control unit controls the display unit to display the biological information of the wearer on the first display element and to display the biological information of the other person on the second display element.

2. (canceled)

3. The accessory according to claim 1, wherein
the obtainment unit obtains the biological information of the other person via a mobile terminal of the wearer.

4. An accessory comprising:
a sensor that detects biological information of a wearer of the accessory;
a storage unit that stores display manner information;
a display unit; and
a control unit having a first operation mode for controlling the display unit to display the biological information of the wearer according to display manner information for the wearer, and a second operation mode for controlling the display unit to display the biological information of the wearer according to second display manner information, wherein
the second display manner information is display manner information for another person, or display manner information calculated from at least display manner information for another person.

5. The accessory according to claim 4, wherein
the second display manner information is calculated from at least the display manner information for the wearer and the display manner information for the other person.

6. The accessory according to claim 5, wherein
the second display manner information is calculated from the biological information of the wearer, biological information of the other person, the display manner information for the wearer, and the display manner information for the other person.

7. The accessory according to claim 6, wherein
the second display manner information is calculated from a biological-information synchronization rate calculated from the biological information of the wearer and the biological information of the other person, and from the display manner information for the wearer and the display manner information for the other person.

8. The accessory according to claim 7, wherein
the second display manner information is calculated from the display manner information for the other person, corresponding to a person who, among a plurality of other people, has the longest duration of a high synchronization rate calculated within a predetermined period, and from the display manner information for the wearer.

9. The accessory according to claim 4, wherein
the second display manner information is calculated from a distance between the wearer and the other person, and from the display manner information for the wearer and the display manner information for the other person.

10. The accessory according to claim 4, wherein
the display manner information for the wearer and the second display manner information are set with a mobile terminal of the wearer.

11. A mobile communication terminal comprising
a transfer unit that obtains biological information of another person from another mobile communication terminal via a network and transfers the biological information of the other person to the accessory according to claim 1 communicatively interconnected with the mobile communication terminal.

12. The mobile communication terminal according to claim 11, wherein
the transfer unit further obtains, from the accessory according to claim 1 communicatively interconnected with the mobile communication terminal, biological information of a wearer of the accessory, and transfers the biological information of the wearer to the other mobile communication terminal via the network.

13. A mobile communication terminal comprising
a setting unit that obtains display manner information for another person from another mobile communication terminal via a network and sets, for the accessory according to claim 4 communicatively interconnected with the mobile communication terminal, second display manner information derived from at least the display manner information for the other person.

14. The mobile communication terminal according to claim 13, wherein
the setting unit further transmits, to the other mobile communication terminal via the network, display manner information for a wearer of the accessory according to claim 4 communicatively interconnected with the mobile communication terminal.

15. A program that causes a computer to function as the transfer unit of the mobile communication terminal according to claim 11.

16. A program that causes a computer to function as the setting unit of the mobile communication terminal according to claim 13.

17. A server comprising
a relay unit that relays biological information between a plurality of the mobile communication terminals according to claim 11 via a network.

18. A server comprising
a relay unit that relays display manner information between a plurality of the mobile communication terminals according to claim 13 via a network.
